(19) United States
(12) Patent Application Publication
(10) Pub. No.: US 2013/0251660 A1
(43) Pub. Date: Sep. 26, 2013

(54) SKIN WETTING COMPOSITIONS AND METHODS

(71) Applicant: ADVANCED BIOCATALYTICS CORP., Irvine, CA (US)

(72) Inventors: Michael G. GOLDFELD, Irvine, CA (US); Andrew H. MICHALOW, Irvine, CA (US); Carl W. PODELLA, Irvine, CA (US); John W. BALDRIDGE, Irvine, CA (US)

(73) Assignee: ADVANCED BIOCATALYTICS CORP., Irvine, CA (US)

(21) Appl. No.: 13/850,931

(22) Filed: Mar. 26, 2013

Related U.S. Application Data
(60) Provisional application No. 61/615,635, filed on Mar. 26, 2012.

(51) Int. Cl. A61K 8/97; A61K 8/64; A61K 8/92; A61Q 5/00; A61Q 19/00

(52) U.S. Cl. CPC A61K 8/97; A61Q 5/00; A61Q 19/00; A61K 8/922; A61K 8/927; A61K 8/64
USPC 424/70.19; 424/744; 424/778; 424/725; 424/537; 514/18.6

(57) ABSTRACT

Disclosed are skin wetting and penetrating compositions and methods, where the compositions comprise the following, or combinations thereof: a surfactant, yeast exo-proteins, a stabilizer, and optionally an oil, humectant, or dermatological active agent.

### FIELD OF THE INVENTION

The present invention is in the field of skin care, oral care, hair care compositions comprising yeast fermentation derived components, and methods of using the same.

### BACKGROUND OF THE DISCLOSURE

Materials derived from natural sources and processes have been shown to have benefits in many fields. Due to the sensitivity of skin, many commercial products attempt to use naturally derived compounds and materials for skin care products. Naturally derived products can be detrimental to skin as can be synthetic products, but there are certain benefits, both real and for marketing reasons, for using naturally derived materials for skin care products.

U.S. Pat. No. 5,665,366 discloses the use of enzymes as a topical agent for prevention of dry skin conditions, dandruff and acne.

U.S. Pat. No. 6,190,678 discloses the use of a combination of conditioning components, in dry form on a cloth substrate that, when wetted by the addition of water, reduces the amount of surfactant needed, providing “effective cleansing using lower, and hence less irritating, levels of surfactant.” The conditioning agent is oil soluble and can be synthetic or naturally derived.

U.S. Pat. Nos. 7,427,690 and 7,572,933 disclose use of metal complexes of Schiff's bases from natural amino acids.

U.S. Pat. No. 8,048,859 teaches the following: “The carrier might also include one or more components that facilitate penetration through the upper stratum corneum barrier to the deeper skin layers. Examples of penetration enhancers include, but are not limited to, propylene glycol, ethoxydiglycol, dimethyl isosorbide, urea, ethanol and dimethyl sulfoxide. Other examples include, but are not limited to, microemulsions, liposomes and nanoemulsions.”

U.S. Pat. No. 8,053,400 teaches that the balance of good cleansing, foaming, skin feel, and low irritation is a delicate balance of surfactants, emollients and other compounds, especially in products with dual cleansing and moisturizing characteristics.

U.S. Patent Application Publication No. 20040043940 discusses the excretion of stress proteins by skin cells in response to a stress and discloses methods to protect the stress proteins produced by skin.

### SUMMARY OF THE INVENTION

Disclosed herein are methods of enhancing wetting, spreading, or uptake of a composition on a surface, the method comprising contacting the surface with the composition, wherein the composition comprises a non-enzymatic, yeast fermentation derived mixture, a surfactant, and a stabilizer. Also disclosed herein are compositions for treatment of skin and hair with enhanced wetting, spreading and uptake, the composition comprising (1) a non-enzymatic, yeast fermentation derived mixture, (2) a surfactant, and (3) a stabilizer.

### BRIEF DESCRIPTION OF THE DRAWINGS

FIG. 1 is a schematic of the test for the efficiency of human skin wetting and solution uptake using contact angle and drop volume/shape analysis.

FIG. 2 is a graph showing the contact angle of the solutions in Examples 1 to 8, on human skin, as function of time.

FIG. 3 is a graph showing the drop volume of the solutions in Examples 1 to 8, on human skin, as function of time.

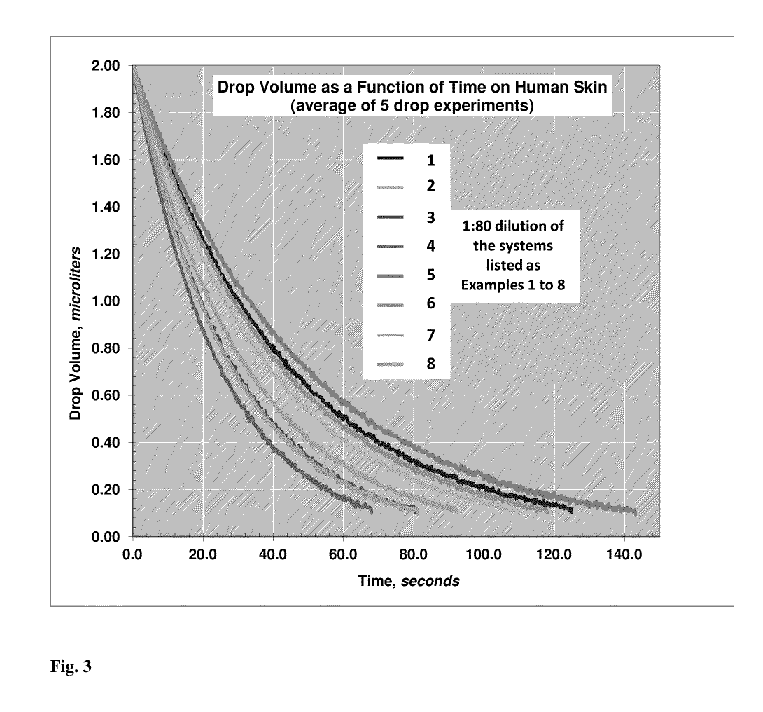

### DETAILED DESCRIPTION OF THE EMBODIMENTS

Yeast extracts disclosed herein, hereinafter, non-enzymatic yeast exo-proteins, were developed to take advantage of what was found to be a synergy between certain yeast exo-proteins when combined with surfactants in wetting of surfaces, enhanced foaming and rinsability.

For the purposes of the present disclosure, the term “surfaces” refers to a biological surface, such as the surface of a mammalian organ or tissue, for example, skin, mucous membrane, nail, or hair. The function of the surfactant is to provide one or more of the following: reducing surface and interfacial tension of solutions, solubilization and emulsification of hydrophobic compounds, improving wetting, spreading, and penetration, enhancing cleansing power or detergency. The term “skin care” comprises both cosmetic and dermatological (i.e., medical or therapeutical) applications and can comprise wetting, penetration, skin uptake or combinations thereof. Most such products contain active ingredient(s) and a carrier. The wetting effectiveness and the level of uptake by the skin depends on many factors, such as the material used in the products, but also the skin condition, the age and health of the user, among others.

Enhancing wetting properties of a skin cleanser improves the cleaning abilities of a cleanser. Good foaming is preferable for consumer appeal. These are at least two characteristics by which the exo-proteins disclosed herein improve on the current state-of-the-art for skin washing agents. The addition of yeast exo-proteins help to improve, or maintain the condition of the skin, by reducing the relative amount of surfactant needed to perform a certain amount of cleaning.

### Yeast Extracts

Yeast extracts have been long known for their use in skin care as live yeast cell derivative, or LYCD, as per Sperti in U.S. Pat. Nos. 2,320,478 and 2,320,479, using an alcohol extraction process with baker's yeast that kills the yeast cells used for extraction by a combination of alcohol treatment and temperature lysis. Such extracts from dead and destroyed cells may contain any proteins from the cell interior, including active enzymes. In contrast, the present disclosure relates to extracts that do not comprise the yeast be killed, and instead, uses exo-proteins that are released by yeast as a response to stress, while maintaining yeast cells alive.

A number of known processes can be used to produce yeast extract, in the course of either aerobic or anaerobic fermentation. Virtually any carbohydrate and nutrient combinations that allow yeast to grow during fermentation can be used. Aerobic processes are preferred due to shorter fermentation times, which can lower costs.

Yeast cells release certain amount of exo-proteins into the external solution during the regular fermentation process and a set of stress proteins in response to various stress conditions, such as heat shock, starvation, radiation, chemical, mechanical stress, etc. Stress proteins are formed and released into the medium by living cells due to the stress-induced expression of certain genes encoding these proteins. Stress proteins are produced by the cells due to the stress-induced expression of certain genes as a response to chemical, thermal, radiation, or mechanical stress that causes certain genes to be expressed by the yeast, therefore stimulating their production of compounds in a fermentation process that can be either anaerobic or aerobic.

In particular, heat has been shown to be a simple, repeatable source of stress for yeast exo-protein production. The processes for the production of stress proteins, and in particular heat shock proteins, is described in U.S. Pat. Nos. 7,476,529, 7,645,730, 7,659,237 and 7,759,301. For example, these patents disclose that: “Prior to centrifugation, the yeast in the fermentation product is subjected to heat-stress conditions by increasing the heat to between 40 and 60 degrees C., for 2 to 24 hours, followed by cooling to less than 25 degrees C.” The entire disclosure of the above-referenced patents, in particular the discussion on the production of stress proteins (for example, column 3, line 41 to column 4, line 51 of U.S. Pat. No. 7,659,237) is incorporated by reference herein.

The thermal stress can be done at lower or higher temperatures, depending on the overall process and particular strain of yeast being used. Saccharomyces cerevisiae start to die off at and above about 70° C., and it is assumed that at some point near this temperature they would stop excreting any proteins. Heat shock, or stress proteins (5) thus defined, as a particular set of exo-proteins, display properties related to the following:

(a) improving surfactant performance in terms of lowering interfacial tension, surface tension, and critical micelle concentration, and skin penetration.

(b) accelerating primarily aerobic microbial metabolic rates with a mechanism shown to rely, at least partially, on uncoupling of oxidative phosphorylation in bacterial cells.

### Yeast Exo-Proteins

In some embodiments, yeast exo-proteins are produced most economically with aerobic fermentation. However, in other embodiments, anaerobic fermentation can be used as well. In some embodiments, the methods disclosed herein further lower the cost of producing the essential yeast exo-proteins. In most previously described yeast extract production methods, the yeast cells were first killed, either by high temperature, or by treatment with alcohol, or alkali, etc. Since the yeast cells were destroyed before the extraction occurs, these yeast extracts might contain any components from the yeast cell interior, including active enzymes that might display undesirable biological activities, such as being allergens, or show enzymatic activities, such as those of proteases, that must be taken into account when skin care compositions are formulated. Unlike those, the yeast extracts disclosed herein are produced by a mild heat shock process that does not destroy, or kill the yeast cells, and contain non-enzymatic yeast exo-proteins released as a result of a physiological response of living cell to stress conditions, such as mild, non-lethal heat shock.

The source of yeast exo-proteins is from yeast fermentation and can be produced using anaerobic or aerobic processes, including methods of the Assignee of the current invention. Optionally, the yeast can be sourced using spent yeast from beer yeast, baker's yeast, alcohol yeast, sake yeast, and the like.

Disclosed herein are skin treatment compositions that comprise, but are not limited to, yeast exo-proteins, a surfactant, and a stabilizer. The compositions optionally comprise the following or combinations thereof: aloe vera extract, glycerol, hyaluronic acid, argerline, carbomer, a plant derived oil, mineral oil, cocoa butter, beeswax, tripeptide, propylgallat, glutathion, arnica montana extract, allantoin, calendula extract, a fragrance, beta glucan and inosine.

The compositions described herein include one or more surfactants at a wide range of concentration levels. Some examples of surfactants that are suitable for use in the detergent compositions described herein include the following:

Anionic: Sodium linear alkylbenzene sulphonate (LABS); sodium lauryl sulphate; sodium lauryl ether sulphates; petroleum sulphonates; linosulphonates; naphthalene sulphonates, branched alkylbenzene sulphonates; linear alkylbenzene sulphonates; alcohol sulphates.

Cationic: Stearalkonium chloride; benzalkonium chloride; quaternary ammonium compounds; amine compounds.

Non-ionic: Dodecyl dimethylamine oxide; coco diethanol-amide alcohol ethoxylates; linear primary alcohol polyethoxylate; alkylphenol ethoxylates; alcohol ethoxylates;

EO/PO polyol block polymers; polyethylene glycol esters; fatty acid alkanolamides.

Amphoteric: Cocoamphocarboxyglycinate; cocamidopropylbetaine; betaines; imidazolines.

In addition to those listed above, suitable nonionic surfactants include alkanolamides, amine oxides, block polymers, ethoxylated primary and secondary alcohols, ethoxylated alkylphenols, ethoxylated fatty esters, sorbitan derivatives, glycerol esters, propoxylated and ethoxylated fatty acids, alcohols, and alkyl phenols, alkyl glucoside glycol esters, polymeric polysaccharides, sulfates and sulfonates of ethoxylated alkylphenols, and polymeric surfactants. Suitable anionic surfactants include ethoxylated amines and/or amides, sulfosuccinates and derivatives, sulfates of ethoxylated alcohols, sulfates of alcohols, sulfonates and sulfonic acid derivatives, phosphate esters, and polymeric surfactants. Suitable amphoteric surfactants include betaine derivatives. Suitable cationic surfactants—include amine surfactants. Those skilled in the art will recognize that other and further surfactants are potentially useful in the compositions depending on the particular detergent application.

Preferred anionic surfactants used in some detergent compositions include CalFoam™ ES 603, a sodium alcohol ether sulfate surfactant manufactured by Pilot Chemicals Co., and Steol™ CS 460, a sodium salt of an alkyl ether sulfate manufactured by Stepan Company. Preferred nonionic surfactants include Neodol™ 25-7 or Neodol™ 25-9, which are C₁₂-C₁₅ linear primary alcohol ethoxylates manufactured by Shell Chemical Co., and Genapol™ 26 L-60, which is a C₁₂-C₁₆ natural linear alcohol ethoxylated to 60E C cloud point (approx. 7.3 mol), manufactured by Hoechst Celanese Corp.

Several of the known surfactants are non-petroleum based. For example, several surfactants are derived from naturally occurring sources, such as vegetable sources (coconuts, palm, castor beans, etc.). These naturally derived surfactants may offer additional benefits such as biodegradability.

The presently disclosed compositions comprise a stabilizer. In some embodiments, the stabilizer is a protein or enzyme stabilizer. Enzyme and protein stabilizers are well-known in the art. In certain embodiments, the stabilizer is an ether, a boric acid derivative, or a boronic acid derivative. In some embodiments, the ether is a glycol ether, for example propylene glycol. In some embodiments, the stabilizer is borax or a substituted phenyl boronic acid, for example 4-alkylcarbonylphenyl boronic acid, where alkyl is a C₁-C₆ alkyl, such as methyl, ethyl, propyl, n-butyl, isobutyl, and or -butyl.

In some embodiments, the compositions disclosed herein comprise between about 0.01% and about 50% by volume of surfactant. In other embodiments, the compositions disclosed herein comprise between about 0.1% and about 30%, or between about 1% and about 25% by volume of surfactant.

In some embodiments, the compositions disclosed herein comprise between about 0.01% and about 20% by volume of the yeast exo-protein mixture. In other embodiments, the compositions disclosed herein comprise between about 0.1% and about 15%, or between about 1% and about 10% by volume of the yeast exo-protein mixture.

In some embodiments, the compositions disclosed herein comprise between about 0.01% and about 50% by volume of stabilizer. In other embodiments, the compositions disclosed herein comprise between about 0.1% and about 30%, or between about 1% and about 25% by volume of stabilizer.

Throughout the present disclosure the term “about” a certain value means that a range of value±10%, and preferably a range of value±5%, is contemplated. Thus, for example, having about 70% of the surfactant includes the surfactant being present between 63% and 87%, and preferably between 66.5% and 73.5%.

Also disclosed herein are skin treatment compositions that comprise yeast exo-proteins where the exo-proteins improve skin wetting and, depending on the composition, improve penetration of active compounds into the layers of skin.

Also disclosed herein are compositions for a cleaning agent, preferably but not exclusively, for use as a skin or hair cleanser comprising yeast exo-proteins, a surfactant, and a stabilizer, where the exo-proteins have dual function by reducing the amount of surfactant and improve wetting of the surfactant for improved cleansing.

Also disclosed herein are compositions for a cleansing agent, that comprises a surfactant and, where the addition of yeast exo-proteins of the current invention, improves foaming with less surfactant.

In another aspect, disclosed herein are methods of enhancing or improving the wetting, spreading, or uptake of a composition on a surface, the method comprising contacting the surface with the composition, wherein the composition comprises a non-enzymatic, yeast fermentation derived mixture, a surfactant, and a stabilizer. By “enhancing” or “improving” it is meant that the wetting, spreading, or uptake of the composition on a surface is better in the presence of the compositions disclosed herein as compared with in the absence of the compositions disclosed here.

### Skin Wetting Tests

Skin wetting improvements using yeast exo-proteins are shown below.

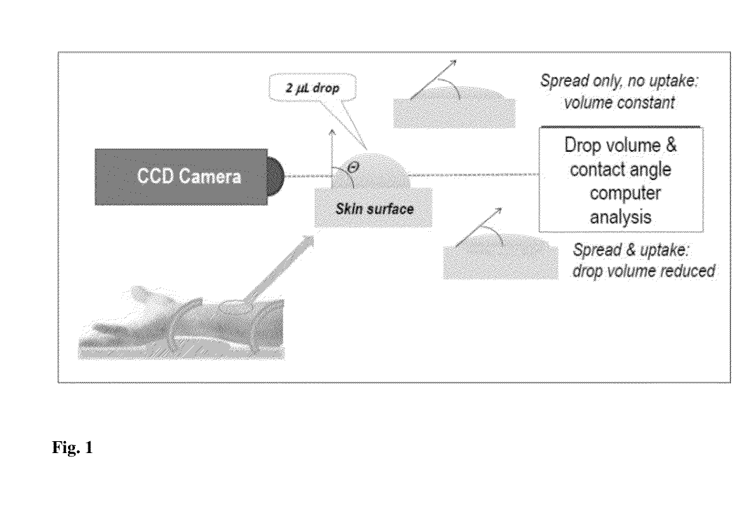

The skin used in the tests was that of a 32 year old Caucasian woman. Five 2.0 microliter drops of each solution of Table 1 was placed on the inside of the forearm and allowed to spread and penetrate into the skin over time. The arm was fixed in a position in front of a Kruss tensiometer camera that records the shape of the droplet, while a specialized software performs analysis of the shape, contact angle and volume of the droplet as a function of time. The scheme of the set is shown in FIG. 1.

The samples were prepared using surfactants specifically designed for skin care application, with and without the yeast exo-protein mixture disclosed herein. All the surfactants were from Stepan Co.: sodium laureth sulfate (Stepanol CS-230), disodium laureth sulfosuccinate (Stepan Mild SL3-BA), (both anionics); and non-ionic co-surfactant lauryl lactyl lactate (Stepan Mild L3).

Table 1 shows the compositions of the solutions used in Examples 1 to 8.

TABLE 1
Compositions of the solutions in Examples 1 to 8.

| | Exam- | Exam- | Exam- | Exam- |
|---|---|---|---|---|
| Ingredient | ple 1 | ple 2 | ple 3 | ple 4 |
| Sodium Laureth | 19.25% | 19.25% | 19.25% | 19.25% |
| Sulfate (26%) | | | | |
| Lauryl Lactyl | 0.00% | 2.75% | 0.00% | 2.75% |
| Lactate | | | | |
| Yeast Exo- | 0.00% | 0.00% | 5.00% | 5.00% |
| Protein | | | | |
| Solution | | | | |
| Water | To | To | To | To |
| | 100.00% | 100.00% | 100.00% | 100.00% |
| | Exam- | Exam- | Exam- | Exam- |
| Ingredient | ple 5 | ple 6 | ple 7 | ple 8 |
| Disodium Lauryl | 15.50% | 15.50% | 15.50% | 15.50% |
| Sulfosuccinate | | | | |
| (32%) | | | | |
| Lauryl Lactyl | 0.00% | 2.21% | 0.00% | 2.21% |
| Lactate | | | | |
| Yeast Exo- | 0.00% | 0.00% | 5.00% | 5.00% |
| Protein | | | | |
| Solution | | | | |
| Water | To | To | To | To |
| | 100.00% | 100.00% | 100.00% | 100.00% |

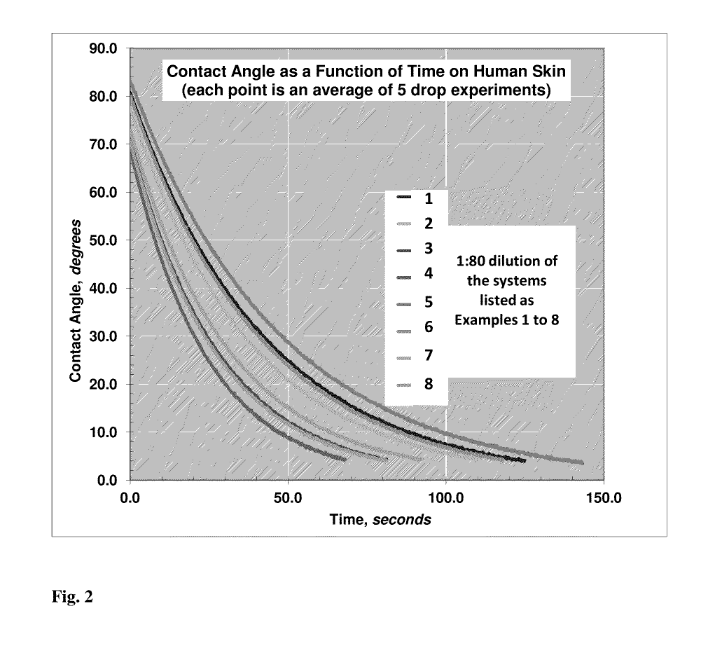

The kinetics of the droplet evolution, presented in terms of contact angle and volume decrease, is shown in FIGS. 1 and 2.

In Table 2, the results are presented as contact angles at the droplet onset, and then at the 95% uptake, and also as time necessary for the uptake of 95% of the droplet volume. The samples are coupled, with and without yeast exo-protein, the upper sample number being without proteins (served as control), and the bottom sample with yeast exo-protein (+Pr).

TABLE 2

| | 80:1 (v/v) Dilution in Water of the mixtures listed in Table 1 | | |
|---|---|---|---|
| | Initial Contact | Time to 95% | Contact Angle |
| | Angle on Skin | Drop uptake | at 95% Uptake |
| | (average of 5 | (average of 5 | (average of 5 |
| | drops) (degrees) | drops) (seconds) | drops) (degrees) |
| Example 1 | 80.8 | 125.0 | 4.1 |
| Example 3 | 71.0 | 81.2 | 4.3 |
| (+Pr) | | | |
| Example 2 | 77.8 | 109.8 | 4.2 |
| Example 4 | 69.1 | 68.0 | 4.3 |
| (+Pr) | | | |
| Example 5 | 83.2 | 143.2 | 3.4 |
| Example 7 | 73.0 | 92.4 | 4.3 |
| (+Pr) | | | |
| Example 6 | 79.9 | 118.0 | 4.3 |
| Example 8 | 70.7 | 81.0 | 3.9 |
| (+Pr) | | | |

On the average, the rate of uptake to 95% drop penetration is about 35% faster in the samples that included the protein mixture. It is a feature of the presently disclosed compositions and methods that the yeast exo-proteins could be used to improve topical skin moisturizing lotions and creams and therapeutic topical skin treatments.

The comparisons between the different surfactant-only packages are less dramatic than the addition of the proteins. Example 5 has the slowest penetration rate at 143.2 seconds for 95% sorption. When Lauryl lactyl lactate is added to it, as with sample Example 6, the time drops 17.6% to 118.0 seconds.

Example 1 (no yeast exo-protein) has a 95% penetration time at 125.0 seconds. When Lauryl lactyl lactate is added to it, as with Example 2, the time drops 12.2% to 109.8 seconds.

In both cases the proteins had more impact on penetration rate, and still had the 31% to 38% impact even after the Stepan Mild L3 addition, as if acting independently.

### REFERENCES

1. Stewart, G G; Russell, I (1998). “Brewer's Yeast”. Brewing Science & Technology Series III (The Institute of Brewing, London).
2. Arch Surg. 1984; 119(9):1005-1008. Acceleration of Wound Healing by a Live Yeast Cell Derivative Jerold Z. Kaplan, M D
3. An Introduction to Brewing Science & Technology Series III, Brewer's Yeast “The IBD Blue Book on Yeast” Institute of Brewing and Distilling
4. Appl Environ Microbiol. 1999 July; 65(7): 3261-3263 Development of Bacterial Contamination during Production of Yeast Extracts—Julie Barrette,¹ Claude P. Champagne,²* and Jacques Goulet¹
5. Heat shock proteins: modifying factors in physiological stress responses and acquired thermotolerance. Kevin C. Kregel (2001) J. Applied Physiol. v. 92 (5), pp. 2177-2186

The following US patents are also referenced:
U.S. Pat. Nos. 2,320,478, 2,320,479, 3,404,068, 3,635,797, 4,017,641, 4,537,776, 4,552,872, 4,557,934, 4,575,457, 4,942,031, 5,238,925, 5,356,873, 5,514,591, 5,643,587, 5,656,300, 5,665,366, 5,676,956, 5,714,169, 5,776,441, 6,190,678, 6,342,208, 6,858,212, 7,186,754, 7,300,649, 7,427,690, 7,455,848, 7,524,816, 7,572,933, 7,666,397, 7,736,633, 7,759,460, 7,777,073, 7,790,147, 7,833,956, 7,851,518, 7,959,935, 8,048,859, 8,053,400, 20040043940

What is claimed is:

1. A method of enhancing wetting, spreading, or uptake of a composition on a surface, the method comprising contacting the surface with the composition, wherein the composition comprises a non-enzymatic, yeast fermentation derived mixture, a surfactant, and a stabilizer.

2. The method of claim 1, wherein the surface is a biological surface.

3. The method of claim 1, wherein the surface is skin or hair.

4. The method of claim 1, wherein the yeast fermentation derived mixture comprises at least one yeast exo-protein.

5. The method of claim 1, wherein the yeast is Saccharomyces cerevisiae.

6. The method of claim 1, wherein the surfactant comprises two or more surfactants.

7. The method of claim 1, wherein the fermentation process is aerobic.

8. The method of claim 1, wherein the surfactant comprises an anionic, a non-ionic, or an amphoteric surfactant, or a combination thereof.

9. The method of claim 1, wherein the composition further comprises one or more ingredients selected from the group consisting of aloe vera extract, calendula extract, glycerol, hyaluronic acid, argeriline, carbomer, plant oil, beeswax, a vitamin, an antioxidant, a fragrance, inosine, and beta glucan.

10. The method of claim 1, wherein the yeast fermentation derived mixture comprises heat shock proteins.

11. A composition for treatment of skin and hair with enhanced wetting, spreading and uptake, the composition comprising (1) a non-enzymatic, yeast fermentation derived mixture, (2) a surfactant, and (3) a stabilizer.

12. The composition of claim 11, wherein the yeast fermentation derived mixture comprises at least one yeast exo-protein.

13. The composition of claim 11, wherein the yeast is Saccharomyces cerevisiae.

14. The composition of claim 11, wherein the surfactant comprises two or more surfactants.

15. The composition of claim 11, wherein the fermentation process is aerobic.

16. The composition of claim 11, wherein the surfactant comprises an anionic, a non-ionic, or an amphoteric surfactant, or a combination thereof.

17. The composition of claim 11, wherein the composition further comprises one or more ingredients selected from the group consisting of aloe vera extract, calendula extract, glycerol, hyaluronic acid, argeriline, carbomer, plant oil, beeswax, a vitamin, an antioxidant, a fragrance, inosine, and beta glucan.

18. The composition of claim 11, wherein the yeast fermentation derived mixture comprises heat shock proteins.

19. The composition of claim 11, wherein the stabilizer is selected from the group consisting of propylene glycol and borax.

20. The composition of claim 11, wherein the surfactant is selected from the group consisting of sodium laureth sulfate, sodium lauryl sulfate, lauryl lactyl lactate, and disodium lauryl sulfosuccinate.
